(19) United States
(12) Patent Application Publication
(10) Pub. No.: US 2009/0169619 A1
(43) Pub. Date: Jul. 2, 2009

(54) CARBAMAZEPINE EXTENDED RELEASE DOSAGE FORM

(75) Inventors: Mukteeshwar Gande, East Windsor, NJ (US); Rasik Gondalia, Edison, NJ (US); Madhusudanarao Kothapalli, Plainsboro, NJ (US); Naga Mahendar Velishala, Piscataway, NJ (US); Vamsi Koppuri, Piscataway, NJ (US)

Correspondence Address: GIBSON & DERNIER L.L.P., 900 ROUTE 9 NORTH, SUITE 504, WOODBRIDGE, NJ, 07095, US

(73) Assignee: CorePharma LLC, Middlesex, NJ (US)

(21) Appl. No.: 12/400,513

(22) Filed: Mar. 9, 2009

Related U.S. Application Data
(63) Continuation of application No. 11/359,813, filed on Feb. 22, 2006.
(60) Provisional application No. 60/656,294, filed on Feb. 25, 2005.

(51) Int. Cl. A61K 9/52; A61K 31/55; A61P 25/14

(52) U.S. Cl. 424/458; 514/217

(57) ABSTRACT

Extended release pharmaceutical dosage forms of carbamazepine for oral administration to maintain a patient's blood concentration for at least a 12 hour period, methods of administering dosage forms and processes for the preparation of such dosage form.

### CROSS REFERENCE TO RELATED APPLICATION

This is a continuation of U.S. application Ser. No. 11/359,813 filed on Feb. 22, 2006, which claims the benefit of U.S. Provisional Patent Application No. 60/656,294, filed Feb. 25, 2005, entitled “Carbamazepine Extended Release Dosage Form,” the entire disclosures of which are hereby incorporated by reference.

### BACKGROUND OF THE INVENTION

The present invention relates to the field of extended release pharmaceutical dosage forms, and in particular to extended release dosage forms of carbamazepine for oral administration and to processes for the preparation of such dosage forms.

Assessment of risk to benefit ratio in patients with epilepsy is crucial in determining the need for treatment, the choice of drugs and the use of monitoring tools such as laboratory tests and other investigations. Active epilepsy per se carries significant risks in terms of increased mortality, susceptibility to psychopathology and physical injury, and reduced quality of life as a result of restricted lifestyle, stigma and prejudice. By preventing the occurrence of seizures, antiepileptic drugs (AEDs) attenuate or eliminate altogether seizure-related risks, but other risks may arise due to the side effects of the drugs, all of which have a relatively Narrow Therapeutic Index (NTI).

NTI drugs are agents for which small changes in systemic concentration can lead to significant changes in pharmacodynamics response. This may result in potentially sub therapeutic or toxic effects, particularly in patients with advanced age, comorbid illness, or those receiving multiple medications. Pharmacologic agents with a therapeutic index <2 are classified as NTI drugs. One such NTI drug is carbamazepine.

Carbamazepine is an iminostilbene derivative that is used clinically to treat seizure disorders and trigeminal neuralgia. It is chemically 5H-Dibenz[b,f]azepine-5-carboxamide. It has poor solubility in water. In the market it is available in different oral dosage forms including extended release tablets and capsules.

U.S. Pat. Nos. 5,326,570 and 5,912,013 describe the composition and method of treating a patient by administering carbamazepine in a pharmaceutical dosage form consisting of three different types of units: immediate-release pellets, sustained release pellets, and enteric release pellets filled in a hard gelatin capsule. This dosage form purports to maintain the patient's blood concentration from about 4 μg/mL to about 12 μg/mL, which is within the therapeutic range required for the treatment of epilepsy as well as other psychiatric, neurological and other disorders.

Processing of this multiple unit dosage form is difficult, time consuming, and costly, requiring quality controls of three separate and distinct units to achieve a final product of the desired quality. Further, undesired effects in any of the units will affect the final product quality thereby diminishing the desired therapeutic effect and the aim of fewer side effects.

Extended release dosage forms are specifically designed to increase the therapeutic window for the patient receiving the treatment. The therapeutic window is the amount of time the blood drug concentration remains below the toxicity range and above the minimum effective concentration. Immediate release formulations have a relatively short therapeutic window but extended release formulations increase that window, resulting in fewer side effects.

The ability to administer a single dose of medication which releases active ingredient over an extended period of time as opposed to the administration of a number of single doses at regular intervals is advantageous to both patient and clinician. In most such dosage preparations, active pharmaceutical agents are either coated with varying thicknesses of some type of relatively insoluble material or are imbedded into a rigid lattice of resinous material. In such preparations, the object is to continuously provide drug for absorption into the bloodstream to replace the amount eliminated while the dosage form is passing through the gastrointestinal tract of the patient.

### SUMMARY OF THE INVENTION

It is an object of the present invention to provide an extended release dosage form for oral administration comprising carbamazepine.

It is another object of the present invention to provide an efficient process for manufacturing a single unit extended release pharmaceutical dosage form.

It is still a further object of the present invention to provide a process for the preparation of a carbamazepine extended release pharmaceutical dosage form for oral administration which comprises the steps of a) combining Carbamazepine with other pharmaceutical excipients and organic solvent, water or hydra alcoholic mixture; b) coating the blend a) with polymer; and c) formulating the coated blend b) into a dosage form.

It is a further object of the present invention to provide a method of treating epilepsy in human patients, comprising an effective dose of carbamazepine or a pharmaceutically acceptable salt thereof and a controlled-release carrier to control the release of said carbamazepine or pharmaceutically acceptable salt thereof.

### BRIEF DESCRIPTION OF THE DRAWINGS

FIG. 1 is a graphical representation of a comparative dissolution profile of formulations in accordance with the present invention.

FIGS. 2-3 are graphical representations of data relating to biostudies employing formulations in accordance with the present invention.

### DETAILED DESCRIPTION OF THE PREFERRED EMBODIMENTS

In the following description, for purposes of explanation, specific numbers, materials and configurations are set forth in order to provide a thorough understanding of the invention. It will be apparent, however, to one having ordinary skill in the art, that the invention may be practiced without these specific details. In some instances, well-known features may be omitted or simplified so as not to obscure the present invention. Furthermore, reference in the specification to “one embodiment” or “an embodiment” means that a particular feature, structure or characteristic described in connection with the embodiment is included in at least one embodiment of the invention. The appearances of the phrase “in an embodiment” in various places in the specification are not necessarily all referring to the same embodiment.

The present invention provides novel extended release formulations for oral administration containing Carbamazepine and excipients.

In accordance with at least one embodiment of the present invention an oral administration dosage form containing Carbamazepine is provided, the formulation further containing at least one pharmaceutically acceptable controlled-release polymer and optionally other pharmaceutically acceptable excipients. Preferably, compositions of the invention comprise a) a granulate containing Carbamazepine and optionally other excipients and b) a controlled-release coating containing at least one pharmaceutically acceptable controlled-release polymer and optionally a plasticizer.

### Granulate

Granulates in accordance with the present invention contain between 100-400 mg of carbamazepine and excipients. Preferably, granulates in accordance with the present invention contain 10-60 mg of diluent, 10-130 mg of retarding polymer and 1-10 mg of binder.

Excipients in accordance with the present invention may be selected from among diluents, binders, retarding polymers, lubricants, and coloring agents which are chemically and physically compatible with Carbamazepine.

Diluents in accordance with the present invention may be selected from any such pharmaceutically acceptable excipients, which give bulk to the Carbamazepine composition, such as but not limited to starch, microcrystalline cellulose, lactose, Mannitol, glucose, fructose, maltose, calcium phosphate and calcium carbonate. Lactose is a preferred Diluent.

Binders include but are not limited to any pharmaceutically acceptable excipient having cohesive properties to act as binders to provide desired granules. Preferably, these binders are selected from one or more of corn starch, Polyvinylpyrrolidone, Hypromellose, gelatin, Ethylcellulose, Pregelatinized starch, Hydroxypropyl cellulose, acrylic acid derivatives, gums, and polyethylene glycols. These materials may be useful individually or in combination with one another as binders and matrix formers for the purpose of this invention.

Retarding polymers in accordance with the present invention used in the granulation process may be selected from either hydrophilic or hydrophobic polymers individually or in combination with one another, such as but not limited to any pharmaceutically acceptable polymer such as Hydroxypropyl cellulose, Hydroxypropyl methylcellulose, Hydroxypropyl Ethylcellulose, Hydroxyethyl cellulose, Ethylcellulose, methylcellulose, Carboxymethyl cellulose, nitrocellulose, copolymers of Ethacrylic acid and methacrylic acid, Carbopol, Hypromellose phthalate, cellulose acetate phthalate, sodium Carboxymethyl methyl cellulose, sodium Carboxymethyl ethyl cellulose and the like. In an embodiment, a combination of Hypromellose and Ethylcellulose is employed as retarding polymer.

Hypromellose polymers are water soluble polymers derived from cellulose, the most abundant polymer in nature. Hypromellose as the controlled release agent in hydrophilic matrix systems offers a wide range of properties.

Lubricants in accordance with the present invention may be selected from pharmaceutically acceptable lubricants including but not limited to talc, magnesium stearate, calcium stearate, zinc stearate, stearic acid, polyethylene glycol, sodium lauryl sulfate, magnesium lauryl sulfate, hydrogenated vegetable oil, sodium stearyl fumarate and the like. In an embodiment, magnesium stearate is employed as a lubricant.

Coloring agents can be used in accordance with the present invention and may be selected from any pharmaceutically acceptable colorant, including those approved and/or certified by the FDA. Colorants include but are not limited to Lake of D&C yellow #11, FD&C yellow #6, #5, FD&C red #3, #4, #40, FD&C blue #1 and the like, individually or in combination with one another.

### Coating

Controlled release coatings in accordance with the present invention contain at least one pharmaceutically acceptable controlled-release coating polymer and optionally a plasticizer.

In accordance with one aspect of the invention a controlled release coating polymer is provided in an amount that is between 0.3%-10% by weight of the granulate. In another embodiment a plasticizer is employed in the coating in an amount that is between 10% to 35% by weight of the coating polymer.

Controlled release coating polymers in accordance with the present invention can be selected from any pharmaceutically acceptable polymer such as but not limited to a high number average molecular weight release controlling polymer, Hydroxypropyl cellulose, Hydroxypropyl methylcellulose, Hydroxypropyl Ethylcellulose, hydroxyethyl cellulose, Ethylcellulose, methylcellulose, Carboxymethyl cellulose, nitrocellulose, copolymers of Ethacrylic acid and methacrylic acid, carbopol, Hypromellose phthalate, cellulose acetate phthalate, sodium Carboxymethyl methyl cellulose, sodium Carboxymethyl ethyl cellulose and the like, individually or in combination with one another. Coating polymers may be hydrophilic or hydrophobic. In an embodiment, Ethylcellulose is employed as a coating polymer. Ethylcellulose polymers are a family of organosoluble thermoplastics that have been widely used in pharmaceuticals.

Plasticizers in accordance with the present invention may be selected from any pharmaceutically acceptable plasticizer including but not limited to tributyl citrate, triethyl citrate, triacetin, dibutyl sebacate, dibutyl phthalate, diethyl phthalate, dimethyl phthalate, acetylated monoglycerides, benzyl benzoate, oleic acid, stearic acid, stearyl alcohol, castor oil, corn oil, refined mineral oils, acetyltributyl citrate, acetyltriethyl citrate and the like, individually or in combination with one another. In an embodiment, triacetin is employed as a plasticizer.

Organic solvents used for granulation and coating can be selected from suitable organic solvents including but not limited to isopropyl alcohol, ethanol, chloroform, methyl acetate, tetrahydrofuran, ethyl acetate, methanol, toluene and the like, individually or in combination with one another. In an embodiment, ethanol is employed as an organic solvent.

In one embodiment granulates in accordance with the invention are coated prior to tabletting. In another embodiment granulates are tabletted prior to coating. In another embodiment granulates are coated and filled in capsules.

In one embodiment the oral dosage form is provided in a single unit extended release form such as but not limited to a hard gelatin capsule, tablet or other suitable pharmaceutical dosage form.

In a most preferred embodiment, compositions in accordance with the present invention contain the following ranges of quantities of carbamazepine and excipients:

Granulate

| | |
|---|---|
| Carbamazepine | 100-400 mg |
| Lactose | 10-60 mg |
| Hypromellose | 10-50 mg |
| Ethylcellulose | 10-80 mg |
| Povidone | 1-10 mg |

Controlled Release Coating polymer

| | |
|---|---|
| Ethylcellulose | 0.3%-10% of |
| | Granules/Pellets |
| Triacetin | 10-35% of |

### Ethylcellulose

The present invention also provides a process for the preparation of a carbamazepine extended release pharmaceutical dosage form for oral administration which includes steps of a) combining carbamazepine with other pharmaceutical excipients and organic solvent, water or hydro alcoholic mixture; b) coating the blend a) with polymer; and c) formulating the coated blend into a dosage form such as but not limited to a tablet, hard gelatin capsule or the like.

The term ‘combining’ includes mixing and/or granulating carbamazepine or a carbamazepine containing mixture, and pharmaceutical excipients, with a sufficient amount of organic solvent, water or hydro-alcoholic mixture, preferably an organic solvent and most preferably ethanol.

In one embodiment, the combining step is accomplished by granulating a blend of carbamazepine and other pharmaceutical agents with organic solvent, water, or hydro-alcoholic mixture.

In accordance with another embodiment, the combining step is accomplished by granulating carbamazepine with a retarding polymer solution in organic solvent.

In a preferred embodiment a process in accordance with the present invention includes the steps of:

a) Granulating carbamazepine, polymer(s), diluent(s), and binder using an organic solvent such as ethanol.
b) Drying wet granules by a technique selected from tray drying, spray drying, air drying or other suitable drying technique.
c) Milling, screening and/or grinding granules to achieve a desired granular size, preferably in the range of from about 60μ to about 1200μ.
d) Coating of the granules with controlled release coating polymer/ethanol solution.
e) If necessary, sifting the granules and/or lubricating the granules.
f) Forming granules into a desired dosage form such as a tablet, hard gelatin capsule or the like.

As will be apparent to those having skill in the art, granulation processes in accordance with the present invention may be simple granulation followed by sieving, extrusion and marumerization, rotogranulation, or any agglomeration process which results in a granule of reasonable size, preferably in the range of about 60μ to about 1200μ.

It is noted that the use of ethanol as a granulating agent helps in solubility of the rate controlled polymer in the carbamazepine blend, thereby resulting in the extended release carbamazepine-containing granules suitable for coating.

In a preferred embodiment, the coating step employs ethylcellulose in ethanol solvent. This step can be performed in a conventional coating pan or fluid bed coater. Preferably, the concentration of ethylcellulose to that of granules can vary between about 0.3% and about 10% by weight, most preferably in the range of from about 0.5% to about 1.5%. the quantity of ethanol required to make an ethylcellulose solution in accordance with this embodiment may vary but it is preferred that a final concentration of the solution is between about 2 to about 5%, and most preferably between about 4% to about 5%.

In a most preferred embodiment, the coating step employs ethylcellulose having 20% by weight of a suitable plasticizer in ethanol solvent.

### EXAMPLES

The following examples and experiments will serve to further typify the nature of the invention, but should not be construed as a limitation on the scope thereof, which is defined solely by the appended claims.

Examples 1-6 are illustrative formulations in accordance with the present invention to produce suitable granules having characteristics which retard drug release and can withstand coating operation parameters.

### Example 1

| | |
|---|---|
| Carbamazepine | 300 mg |
| Hypromellose | 30 mg |
| Ethylcellulose | 60 mg |
| Lactose | 40 mg |
| Povidone | 5 mg |
| Ethanol | q.s. |

All dry excipients along with carbamazepine were blended for 2 minutes using a high shear mixer granulator. The dry blend was then granulated with ethanol in a high shear mixer granulator. The wet granulated mass was dried in a tray drier. The dried granulate was passed through an ASTM #20 sieve. The sieved granules were then coated in a conventional coating pan using a solution of ethylcellulose having 20% by weight of Triacetin in ethanol solvent to further retard the release rate of carbamazepine. The concentration of ethylcellulose to that of the granules was 1%. The concentration of the ethylcellulose/plasticizer in the ethanol solution was about 5%. The coated granules were sifted through an ASTM #16 sieve and lubricated with magnesium stearate (0.5%). The final granules were filled into pharmaceutically acceptable hard gelatin capsules to produce a final pharmaceutical oral dosage form.

### Prophetic Example 2

| | |
|---|---|
| Carbamazepine | 300 mg |
| Hypromellose | 75 mg |
| Microcrystalline cellulose | 25 mg |
| Lactose | 25 mg |
| Povidone | 5 mg |
| Water | q.s. |

### Prophetic Example 3

| | |
|---|---|
| Carbamazepine | 300 mg |
| Ethylcellulose | 40 mg |
| Hypromellose | 60 mg |
| Dibasic calcium phosphate | 30 mg |
| Water | q.s. |

### Prophetic Example 4

| | |
|---|---|
| Carbamazepine | 300 mg |
| Hypromellose | 80 mg |
| Microcrystalline cellulose | 25 mg |
| Dibasic calcium phosphate | 25 mg |
| Water | q.s. |

### Prophetic Example 5

| | |
|---|---|
| Carbamazepine | 300 mg |
| Hypromellose | 30 mg |
| Ethylcellulose | 60 mg |
| Dibasic calcium phosphate | 40 mg |
| Povidone | 5 mg |
| Ethanol | q.s. |

### Prophetic Example 6

| | |
|---|---|
| Carbamazepine | 300 mg |
| Hypromellose | 35 mg |
| Ethylcellulose | 60 mg |
| Lactose | 40 mg |
| Ethanol | q.s. |

For each of the examples 2-6, processing in general would proceed in accordance with the following steps:

1. Blend all excipients along with carbamazepine for 2-5 minutes using a high shear mixer granulator or other suitable mixing device known in the art.
2. Granulate the dry blend of step 1 with either ethanol or water (as listed in each respective Example) in a suitable mixer or granulator such as but not limited to a high shear mixer granulator or planetary mixer.
3. Dry the wet mass of step 2 either in a suitable drying device such as but not limited to a tray drier or a fluid bed drier.
4. Sieve or mill the dried material of step 3. Sieving can employ an appropriate sieve such as but not limited to an ASTM #20 sieve; milling can employ a suitable mill such as but not limited to a Granu Mill.
5. Coating the granules resulting from step 4 in a suitable coating device such as but not limited to a fluid bed coater using a solution of ethylcellulose having about 20% by weight of plasticizer in an ethanol solvent to further retard the release rate of carbamazepine. Preferably, the concentration of ethylcellulose solution to that of granules is about 1%. Preferably, the concentration of the ethylcellulose/plasticizer in the ethanol solution is about 5%.
6. Preferably, coated granules are sifted through an ASTM #16 sieve and optionally lubricated using an appropriate amount of any pharmaceutically acceptable lubricant, most preferably magnesium stearate.
7. The final granules are either tabletted or filled into pharmaceutically acceptable hard gelatin capsules to make a final pharmaceutical oral dosage form.

### Dissolution

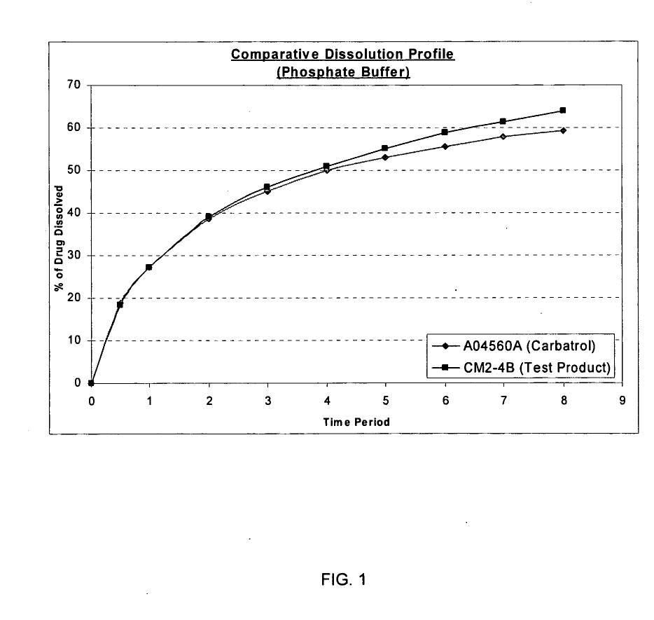

Final granule-filled hard gelatin capsules made in accordance with Example 1 hereinabove were tested for dissolution profile in 900 mL of two different dissolution media: 0.5% Sodium Lauryl Sulfate in water; and in phosphate buffer, using USP apparatus II (Paddle) at paddle speed of 75 rpm. As shown in FIG. 1, for comparison, CARBATROL® 300 mg capsules (carbamazepine extended release capsules) were used. The dissolution profile was studied for an 8 hour period.

The percentage of drug released in 0.5% Sodium Lauryl Sulfate solution achieved in 2 hours was between 50%-70% and in 8 hours was not less than 85%.

The phosphate buffer medium consisted of 50 mL of 0.1N HCl and 850 mL of pH 7.2 phosphate buffer. Now referring to FIG. 1, the average percentage of carbamazepine dissolved in phosphate buffer in 2 hours was between 30%-45% and in 8 hours was between 50%-70%. As can be seen from FIG. 1, the formulations made in accordance with the granulation and coating processes of the present invention provide in-vitro drug release dissolution profiles similar to that of CARBATROL®.

### Bioavailability

As can be seen from the following, bioavailability in-vivo of formulations prepared in accordance with the present invention was compared to CARBATROL®.

### Test Methods

A pilot study was carried out under fasting condition to compare the relative bioavailability of formulations in accordance with the present invention with that of CARBATROL® 300 mg extended release capsules manufactured by Shire US Inc., using a single oral dose (1×300 mg capsule) in healthy adult subjects.

A single-dose, randomized, two-period, two-treatment, two-sequence crossover study design was used to evaluate the relative bioavailability of the carbamazepine extended release capsule formulations of the present invention when dosed under fasting condition.

12 subjects were recruited for this study and all subjects were healthy adults (8 males and 4 females). The plasma samples from 12 subjects were assayed for carbamazepine and carbamazepine 10,11-epoxide.

The pharmacokinetic parameters determined from the plasma concentration data are

a) The area under the plasma concentration versus time curve.
b) The area under the plasma concentration versus time curve from zero to infinity.
c) The maximum observed plasma concentration.
d) The time to maximum plasma concentration.
e) The terminal elimination rate constant.
f) The half-life.

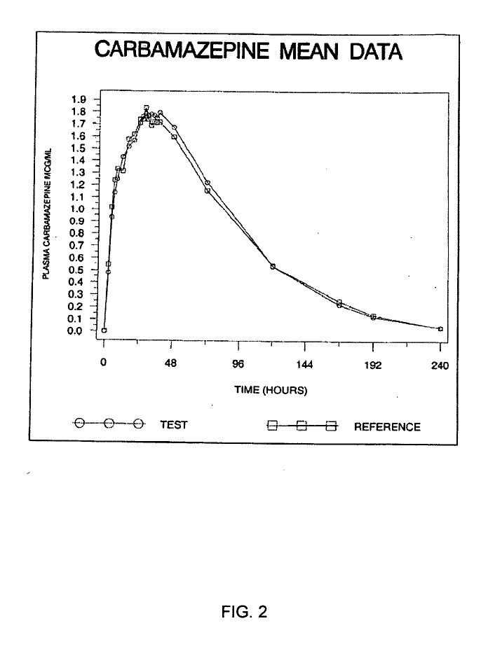

The analytical method was developed and validated and used a high performance liquid chromatography method using a UV detector to determine the concentration of carbamazepine and carbamazepine 10,11-epoxide. As shown in FIG. 2, the analysis of the plasma carbamazepine data resulted in no statistically significant, α=0.05, differences in products for any of the pharmacokinetic parameters.

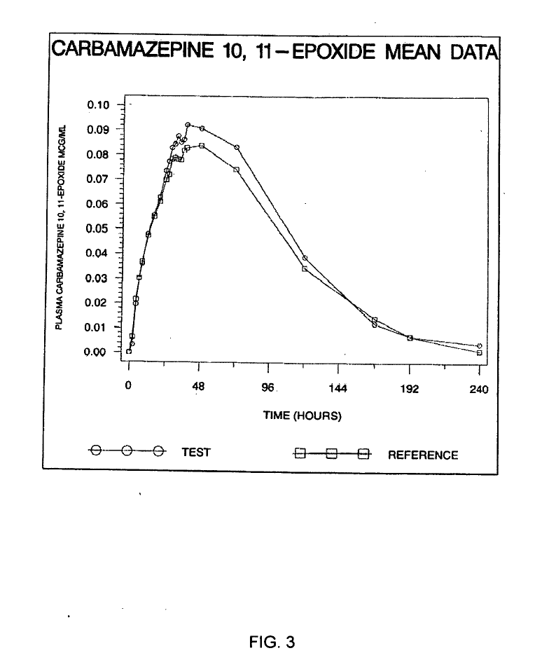

FIG. 3 shows the analysis of the plasma carbamazepine 10,11-epoxide data resulted in no statistically significant, α=0.05, differences in products for any of the pharmacokinetic parameters except AUC∞ (10%), and the log transformed AUC∞ (8%).

The process, in-vitro data and in-vivo data show that novel formulations made in accordance with the present invention employing single process granules are manufactured efficiently with reduced need for quality controls with little variation from batch to batch.

While the present invention has been described in terms of its specific embodiments, certain modifications and equivalents will be apparent to those skilled in the art and are intended to be included within the scope of the present invention.

What is claimed is:

1. A single dosage extended release pharmaceutical composition for oral administration comprising a granulate comprising carbamazepine and at least one pharmaceutically acceptable excipient.

2. A composition in accordance with claim 1 said at least one excipient selected from the group comprising a diluent, a binder, a retarding polymer and a lubricant.

3. A composition in accordance with claim 1 said granulate comprising at least one diluent, at least one binder and at least one retarding polymer.

4. A composition in accordance with claim 3 said granulate further comprising at least one lubricant.

5. A composition in accordance with claim 1 further comprising at least one coating polymer.

6. A composition in accordance with claim 2 further comprising at least one coating polymer.

7. A composition in accordance with claim 4 further comprising at least one coating polymer.

8. A composition in accordance with claim 1 said at least one retarding polymer comprising hypromellose.

9. A composition in accordance with claim 5 said coating polymer comprising ethylcellulose.

10. A formulation comprising the composition in accordance with claim 1 comprising said granulate contained in a capsule.

11. The composition in accordance with claim 1 wherein said composition provides a therapeutically effective dose of carbamazepine over a 12 hour period.

12. The composition in accordance with claim 1 comprising granules comprising 100-400 mg of carbamazepine.

13. The composition in accordance with claim 1 comprising a controlled release coating polymer in an amount of 0.3% to 10% by weight of said granulate.

14. The composition in accordance with claim 1 comprising a high number average molecular weight release controlled release polymer.

15. The composition in accordance with claim 13 said controlled release coating polymer comprising ethylcellulose.

16. The composition in accordance with claim 15 further comprising a plasticizer.

17. A process for the preparation of a single dose carbamazepine dosage form for oral administration comprising the steps of: a) combining carbamazepine with at least one pharmaceutical excipient and an agent selected from the group comprising organic solvents, water and hydroalcoholic mixtures; b) coating the blend a) with at least one polymer; and c) formulating the coated blend b) into a dosage form.

18. A process in accordance with claim 17 comprising employing an organic solvent in said combining step.

19. A process in accordance with claim 17 comprising employing an organic solvent and water mixture in said combining step.

20. A process in accordance with claim 17 further comprising using an organic solvent, water or mixture thereof in said coating step.

21. A pharmaceutical dosage form comprising an active pharmaceutical ingredient comprising a) a granulate containing said active pharmaceutical ingredient and optionally other excipients and b) a controlled-release coating containing at least one pharmaceutically acceptable controlled-release polymer and optionally a plasticizer.

22. A method of treatment of epilepsy in human patients, comprising an effective dose of carbamazepine or a pharmaceutically acceptable salt thereof and a controlled-release carrier to control the release of said carbamazepine or pharmaceutically acceptable salt thereof from said dosage form, said dosage form being suitable for providing once-a-day oral administration of the carbamazepine or pharmaceutically acceptable salt thereof, said oral dosage form comprising a single unit dosage form.
